(19) United States
(12) Patent Application Publication
(10) Pub. No.: US 2006/0216345 A1
(43) Pub. Date: Sep. 28, 2006

(54) Oral pharmaceutical composition including paroxetine

(75) Inventors: Vaishali Vijay Dhavse, Mumbai (IN); Nitin Bhalachandra Dharmadhikari, Mumbai (IN)

Correspondence Address: MERCHANT & GOULD PC, P.O. BOX 2903, MINNEAPOLIS, MN, 55402-0903, US

(73) Assignee: SUN PHARMACEUTICAL INDUSTRIES LIMITED, Mumbai (IN)

(21) Appl. No.: 11/388,725

(22) Filed: Mar. 24, 2006

(30) Foreign Application Priority Data
Mar. 24, 2005 (IN) 336/MUM/2005

(51) Int. Cl. A61K 31/137; A61K 9/48; A61K 9/20

(52) U.S. Cl. 424/451; 424/464; 514/649

(57) ABSTRACT

A stable oral pharmaceutical composition including a therapeutically effective amount of paroxetine or its pharmaceutically acceptable salt and one or more adjuvants that prevent the formation of pink hue upon storage of the composition. The composition can be prepared by aqueous granulation.

### FIELD OF INVENTION

The present invention relates to stable oral pharmaceutical compositions including a therapeutically effective amount of paroxetine or its pharmaceutically acceptable salt(s) and one or more adjuvant(s) that prevent formation of pink hue upon storage of the composition.

### BACKGROUND OF THE INVENTION

Paroxetine is a selective serotonin reuptake inhibitor indicated in the treatment of depression, obsessive compulsive disorder, panic disorder, social anxiety disorder, generalized anxiety disorder and post-traumatic stress disorder. Paroxetine exerts its action by potentiation of serotonergic activity in the central nervous system resulting from inhibition of neuronal uptake of serotonin. It is a potent and highly selective inhibitor of neuronal serotonin reuptake. Paroxetine is commercially available as Paxil® conventional immediate release tablets, and as Paxil® CR controlled release tablets.

U.S. Pat. No. 6,113,944 discloses a pharmaceutical composition in tablet form containing therapeutically effective amounts of paroxetine produced by a process which includes steps of: a) dry admixing paroxetine and excipients in a mixer to form a mixture; or b) dry admixing paroxetine and excipients, compressing the resulting combination into a slug material or roller compacting the resulting combination into a strand material, and milling the prepared material into a free flowing mixture; and c) compressing the mixture into tablets using a single punch or rotary tablet machine. The patent teaches that paroxetine formulated into a tablet using a process in which water is absent, is much less likely to develop a pink hue upon storage.

U.S. Pat. No. 6,645,523 ('523 patent) discloses that coloration problem, i.e. the pink hue, in paroxetine tablets, involves the formation of a coloring impurity identified as compound A which is a dimer formed from paroxetine free base in an aqueous alkaline environment. The '523 patent claims a solid unit dose paroxetine composition including a pharmaceutically effective amount of paroxetine hydrochloride, an acidic calcium phosphate, and a disintegrant, wherein said composition has a pH within the range of 4.5 to 6.0. Thus, the invention discloses a solid paroxetine composition that resists the formation of a colour hue and to processes for making the same with the aid of water, by controlling the pH of the composition to 6.5 or less. Acidic calcium phosphate, as described in the specification of the '523 patent, is a special grade obtained by removing impurities or washing. Commercially available grades of dicalcium phosphate, such as Di-Cafos AN, Anhydrous Emcompress, have a pH greater than 7, and the “Handbook of Pharmaceutical Excipients, 3^{rd} edition, Ed by Arthur H. Kibbe, American Pharmaceutical Association, Washington D.C., 2000” states that these grades should not be used with drugs which are sensitive to alkaline pH. In contrast to the teachings of the '523 patent and the Handbook of Excipients, we have surprisingly found that commercially available grades of dicalcium phosphate having an alkaline pH, can be used to obtain stable compositions of paroxetine.

WO2005034954 ('954 application) discloses a pharmaceutical composition including paroxetine, microcrystalline cellulose and one or more additional pharmaceutical inert excipients, wherein the pharmaceutical composition is prepared by wet granulation technique. In contrast to the teachings of the '954 application, we have surprisingly found that microcrystalline cellulose, in fact, does cause formation of a pink hue when wet granulated with paroxetine.

WO2004091582 describes a moisture barrier coating enveloping the individual granules of the active core, which substantially eliminates the possibility of degradation or colour development. Such a process may be tedious, time consuming and expensive.

### SUMMARY OF THE INVENTION

The present invention provides a stable oral pharmaceutical composition including a therapeutically effective amount of paroxetine or its pharmaceutically acceptable salt(s) and one or more adjuvants that prevent the formation of pink hue upon storage of the composition. In an embodiment, the composition is prepared by aqueous granulation.

### DETAILED DESCRIPTION OF THE INVENTION

It has been surprisingly found that a stable oral pharmaceutical composition including therapeutically effective amounts of paroxetine or its pharmaceutically acceptable salt(s) can be obtained by incorporating one or more adjuvants that prevent the formation of pink hue upon storage of the composition. This can be accomplished using a conventional aqueous granulation process.

The stable oral pharmaceutical composition of the present invention includes therapeutically effective amounts of paroxetine or its pharmaceutically acceptable salt. In an embodiment, the paroxetine is in the hydrochloride form. The paroxetine or its pharmaceutically acceptable salt that may be used in the composition of the present invention may be either in amorphous form or crystalline form, preferably in the crystalline form, most preferably in the crystalline hemihydrate form. The amount of paroxetine or its pharmaceutically acceptable salt that may be used in the composition of the present invention may range from about 1 mg to about 50 mg per unit dosage form, preferably from about 5 mg to about 40 mg, most preferably from about 10 mg to about 40 mg per unit dosage form.

The oral pharmaceutical composition of the present invention includes, but is not limited to, solid dosage forms such as tablets, capsules, sachets, granules and pellets.

The term “stable” as used herein refers to the colour stability of the pharmaceutical composition of paroxetine, wherein the composition when stored does not show any discoloration or formation of pink color/hue. For the purpose of determination of stability, a suitable method may be used. For example, the composition may be stored in high-density polyethylene (HDPA) bottles with child resistant caps at 40° C. and 75% relative humidity for 3 months and observed for development of color. These conditions are believed in the art to be equivalent to storage on the shelf at ambient conditions for a period of two years. The stability may also be determined at other conditions, for example, storage at 60° C. for one month.

The pharmaceutical composition of the present invention includes one or more diluents, flavoring agents, solubilizers, lubricants, suspending agents, binders, tablet disintegration agents, encapsulating materials and the like as adjuvants. Various pharmaceutical compositions were prepared by mixing pharmaceutically acceptable excipients (adjuvants) with paroxetine hydrochloride hemihydrate in a drug to excipient ratio ranging from about 1:0.1 to about 1:1, followed by aqueous granulation. The said compositions were stored at 40° C. at 75% relative humidity for one month, and at 60° C. for a period of one month. The compositions were visually observed for any discoloration or colour formation at the end of 1 month.

The adjuvants (the term excipient and adjuvant have been used interchangeably herein) used in the pharmaceutical composition of the present invention that prevent the formation of the pink hue upon storage of the composition, for example, wherein the composition is prepared by aqueous granulation, are selected from the group consisting of lactose anhydrous, lactose monohydrate, pregelatinized starch, sodium starch glycolate, magnesium stearate, hydroxypropyl methylcellulose, titanium dioxide, polyethylene glycol 6000 and polysorbate 80, silicified microcrystalline cellulose, dicalcium phosphate dihydrate having pH greater than 7.0, dicalcium phosphate anhydrous having pH greater than 7.0, and mixtures thereof.

The amount of compatible adjuvant that may be used may range from about 0.5% by weight to about 90% by weight of the composition, depending upon the function of the adjuvant.

Lactose monohydrate that may be used in the pharmaceutical compositions of the present invention is commercially available as Fast-flo, Lactochem, Microtose, Tablettose, having 4.5-5.5% water content and true density of about 1.552. The lactose monohydrate may be used as diluent in an amount ranging from about 5% to about 50% by weight of the composition.

Lactose anhydrous that may be used as a compatible excipient in the pharmaceutical compositions of the present invention, contains less than 1% of water, and has true density of about 1.552. Preferably, the lactose anhydrous has a bulk density of about 0.67g/cm³, a tapped density of about 0.85g/cm³ and a specific surface area of about 0.35m²/g. The preferred grade of lactose anhydrous is commercially available as Pharmatose DCL 21, having a particle size distribution such that 85% of the particles are less than 250 microns, and 50% of the particles are less than 150 microns. Lactose anhydrous may be used in the pharmaceutical compositions of the present invention in an amount ranging from about 50% to about 90% by weight of the composition.

Dicalcium phosphate anhydrous and dicalcium phosphate dihydrate that may be used as compatible excipients in the pharmaceutical compositions of the present invention have a pH greater than 7.0. They may be used typically as diluents in an amount ranging from about 5% to about 50% by weight of the composition. Preferably, a 20% slurry of the dicalcium phosphate has a pH of about 7.3 to about 7.5. Preferred commercially available dicalcium phosphate anhydrous is Anhydrous Emcompress having an average particle diameter of about 136 microns, and preferred commercially available dicalcium phosphate dihydrate has an average particle diameter of about 180 microns.

Sodium starch glycolate is another compatible excipient that may be used in the pharmaceutical compositions of the present invention as a disintegrant, in an amount ranging from about 0.5% to about 10% by weight of the composition. The preferred grade of sodium starch glycolate has a particle diameter of about 30-100 microns, and a pH of 5.5-7.5 for a 3.3% aqueous dispersion. Commercially available Explotab having an average particle size of 42 microns is the most preferred.

Pregelatinized starch is another compatible excipient that may be used as a diluent, disintegrant or binder in the pharmaceutical compositions of the present invention, in an amount ranging from about 0.5% to about 50% by weight of the composition. Preferably, the pregelatinized starch used has a pH in the range of 4.5 to 7.0 (10% w/v slurry) with particles of size 30-150 microns. Commercially available Starch 1500, having a specific surface area of about 0.26m²/g is the most preferred grade.

Silicified microcrystalline cellulose is yet another compatible excipient that may be used as a diluent or filler in the pharmaceutical compositions of the present invention and is especially suitable for compaction of tablets obtained by the process of wet granulation, such as embodiments of the compositions of the present invention. It may be used in an amount ranging from about 5% to about 50% by weight of the composition. Silicified microcrystalline cellulose that may be used in the compositions of the present invention includes grades that have 2% w/w of colloidal silicon dioxide, and pH in the range of 5.0-7.5. Preferably, the silicified microcrystalline cellulose has a mean particle size of 90 microns, such as that commercially available under the trade name Prosolv SMCC 90. Surprisingly, although microcrystalline cellulose was found to be incompatible with paroxetine or its salts upon wet granulation, silicified microcrystalline cellulose was found to be compatible.

The pharmaceutical composition of the present invention can be obtained by the conventional process of wet granulation, wherein water or a mixture of water with an organic solvent may be used for the process of granulating a mixture including paroxetine or its therapeutically acceptable salt and one or more adjuvants that prevent formation of pink hue upon storage of the composition. The present invention includes a method of making a composition including paroxetine or its therapeutically acceptable salt and one or more adjuvants that prevent formation of pink hue upon storage of the composition. The method can include wet granulating a mixture including paroxetine or its therapeutically acceptable salt and one or more adjuvants that prevent formation of pink hue upon storage of the composition. Wet granulating can employ water or a mixture of water with an organic solvent.

Embodiments of the present invention include:
a. A stable oral pharmaceutical composition including a therapeutically effective amount of paroxetine or its pharmaceutically acceptable salt and one or more adjuvants that prevent the formation of pink hue upon storage of the composition, wherein the composition is prepared by aqueous granulation.
b. A stable oral pharmaceutical composition as described in (a) above, wherein the pharmaceutically acceptable salt of paroxetine is paroxetine hydrochloride hemihydrate.
c. A stable oral pharmaceutical composition as described in (a) above, wherein one or more adjuvants that prevent the formation of pink hue upon storage, are selected from the group including lactose anhydrous, lactose monohydrate, pregelatinized starch, sodium starch glycolate, magnesium stearate, hydroxypropyl methylcellulose, titanium dioxide, polyethylene Glycol 6000 and polysorbate 80, silicified microcrystalline cellulose, dicalcium phosphate dihydrate, dicalcium phosphate anhydrous.
d. A stable oral pharmaceutical composition as described in (a) above, wherein the composition may be in the form of capsule, tablet, pellets or granules.
e. A stable oral pharmaceutical composition as described in (a) above, wherein the composition is stable when stored at 40° C. at 75% relative humidity for 3 months in sealed high-density polyethylene bottles with child resistant caps.
f. A method of making a composition including paroxetine or its therapeutically acceptable salt and one or more adjuvants that prevent formation of pink hue upon storage of the composition, the method including wet granulating a mixture including paroxetine or its therapeutically acceptable salt and one or more adjuvants that prevent formation of pink hue upon storage of the composition, wet granulating employing water or a mixture of water with an organic solvent.

The present invention may be better understood with reference to the following examples. These examples are intended to be representative of specific embodiments of the invention, and are not intended as limiting the scope of the invention.

### EXAMPLE 1

Various pharmaceutical compositions were prepared by mixing pharmaceutically acceptable excipients (adjuvants) with paroxetine hydrochloride hemihydrate in a drug to excipient ratio ranging from about 1:0.1 to about 1:1, followed by aqueous granulation. The said compositions were stored at 40° C. at 75% relative humidity for one month, and at 60° C. for a period of one month. The compositions were visually observed for any discoloration or color formation at the end of 1 month. The results are recorded in Table 1 below.

TABLE 1

| | | Visual colour Observation Results | | |
|---|---|---|---|---|
| | | at various storage conditions | | |
| | Drug to | | 40° C. at | |
| | excipients | | 75% RH for | 60° C. for |
| Excipients | ratio | Initial | 1 month | 1 month |
| Lactose monohydrate | 1:1 | white | No change | No change |
| Lactose anhydrous | 1:1 | white | No change | No change |
| Microcrystalline | 1:1 | white | Slightly Pink | Slightly Pink |
| Cellulose | | | | |
| (comparative example) | | | | |
| Silicified | 1:1 | white | No change | No change |
| Microcrystalline | | | | |
| Cellulose | | | | |
| Dicalcium phosphate | 1:1 | white | No change | No change |
| dihydrate | | | | |
| Dicalcium phosphate | 1:1 | white | No change | No change |
| anhydrous | | | | |
| Hydroxypropyl | 1:0.5 | white | No change | No change |
| Cellulose | | | | |
| Polyvinyl pyrrolidone | 1:0.5 | white | No change | No change |
| (PVP K-30) | | | | |
| Pregelatinized Starch | 1:0.5 | white | No change | No change |
| Magnesium stearate | 1:0.1 | white | No change | No change |
| Sodium starch | 1:0.5 | white | No change | No change |
| glycolate | | | | |
| Opadry (mixture of | 1:0.2 | Yellow | No change | No change |
| Hydroxypropyl | | | | |
| cellulose. Titanium | | | | |
| dioxide, polyethylene | | | | |
| glycol 6000, talc, | | | | |
| iron oxide yellow) | | | | |

### EXAMPLE 2

A pharmaceutical composition including compatible excipients of Example 1 was obtained as mentioned in Table 2 below.

TABLE 2

| Stage of process | Ingredients | mg/tablet | % w/w |
|---|---|---|---|
| Intragranular | Paroxetine hydrochloride | 45.5 | 6.90 |
| | hemihydrate | | |
| | Lactose, anhydrous | 426.5 | 64.7 |
| | Pregelatinized Starch | 64.0 | 9.7 |
| | Lactose, monohydrate | 64.0 | 9.7 |
| Extragranular | Lactose anhydrous | 17.0 | 2.6 |
| | Sodium starch glycolate | 13.0 | 1.97 |
| | Magnesium stearate | 10.0 | 1.5 |
| Coating | Opadry Green 13F51312 | 19.2 | 2.9 |

Paroxetine hydrochloride hemihydrate, lactose monohydrate, pregelatinized starch and lactose anhydrous were sifted through ASTM (American Society for Testing and Materials) 40# mesh. The sifted ingredients were mixed and granulated with water. The granules were dried and milled. The extragranular ingredients namely, lactose anhydrous, sodium starch glycolate and magnesium stearate were sifted and blended with the granules. The lubricated granules were compressed and further film coated with Opadry Green 13F51312.

The film coated tablets were stored at 40° C. at 75% RH in sealed HDPE bottles with child resistant caps. The tablets were broken and the core was observed for the formation of pink hue. Core was found to be white in appearance and no pink hue was observed in the core at the end of three months.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

What is claimed is:

1. A stable oral pharmaceutical composition comprising:
(i) a therapeutically effective amount of paroxetine or its pharmaceutically acceptable salt, and
(ii) one or more pharmaceutically acceptable adjuvants selected from the group consisting of lactose anhydrous, lactose monohydrate, pregelatinized starch, sodium starch glycolate, magnesium stearate, hydroxypropyl methylcellulose, titanium dioxide, polyethylene glycol 6000 and polysorbate 80, silicified microcrystalline cellulose, dicalcium phosphate dihydrate and dicalcium phosphate anhydrous,
wherein the composition is prepared by aqueous granulation.

2. A stable oral pharmaceutical composition comprising:
(i) a therapeutically effective amount of paroxetine or its pharmaceutically acceptable salt, and
(ii) pharmaceutically acceptable adjuvants comprising lactose, pregelatinized starch, sodium starch glycolate and magnesium stearate,
wherein the composition is prepared by aqueous granulation.

3. A stable oral pharmaceutical composition as claimed in claim 1 wherein the pharmaceutically acceptable salt of paroxetine is paroxetine hydrochloride hemihydrate.

4. A stable oral pharmaceutical composition as claimed in claim 1 wherein the composition may be in the form of capsule, tablet, pellets or granules.
